(19) United States
(12) Patent Application Publication
(10) Pub. No.: US 2025/0307367 A1
(43) Pub. Date: Oct. 2, 2025

(54) INFORMATION PROCESSING SYSTEM, INFORMATION PROCESSING METHOD, AND NON-TRANSITORY COMPUTER READABLE MEDIUM

(71) Applicant: NEC Corporation, Tokyo (JP)

(72) Inventors: Yuki TANAKA, Tokyo (JP); Hiroto TAMIYA, Tokyo (JP); Daichi AOKI, Tokyo (JP); Takuya HAYASHI, Tokyo (JP); Kengo MORI, Tokyo (JP); Toshiyuki ISSHIKI, Tokyo (JP)

(73) Assignee: NEC Corporation, Tokyo (JP)

(21) Appl. No.: 19/075,026

(22) Filed: Mar. 10, 2025

(30) Foreign Application Priority Data
Mar. 27, 2024 (JP) 2024-051938

(51) Int. Cl. G06F 21/32; G16H 10/60

(52) U.S. Cl. CPC G06F 21/32; G16H 10/60

(57) ABSTRACT

The identification of a patient is checked more reliably. An information processing system includes: biometric information acquisition means for acquiring biometric information of a patient; authentication processing means for authenticating the patient based on the biometric information acquired by the biometric information acquisition means; first storage means for storing the biometric information of the patient authenticated by the authentication processing means in association with identification information of the patient; trace information generation means for generating signature information of the patient based on the biometric information of the patient authenticated by the authentication processing means and generating signed trace information containing the signature information; trace information acquisition means for acquiring the signed trace information generated by the trace information generation means; and verification processing means for performing a verification process on the signature information of the signed trace information acquired by the trace information acquisition means.

### INCORPORATION BY REFERENCE

This application is based upon and claims the benefit of priority from Japanese patent application No. 2024-051938, filed on Mar. 27, 2024, the disclosure of which is incorporated herein in its entirety by reference.

### TECHNICAL FIELD

The present disclosure relates to an information processing system, an information processing method, and a program.

### BACKGROUND ART

For example, at a medical institution, a specimen such as blood or a part of a tumor of a cancer is taken from a patient, and based on the taken specimen, a so-called personalized vaccine specialized for that patient is generated through a plurality of institutions and companies. Then, the generated personalized vaccine is administered to the same patient at a medical institution (which is the same as or different from the aforementioned medical institution). In regard to such a vaccine generation flow between a medical institution at which a specimen is detected and a medical institution at which a personalized vaccine is administered, a system for managing a product(s) in such a flow is known (see, for example, Patent Literature 1).

Patent Literature 1: International Patent Publication No. WO2023/058625

However, in the case where management is performed only between the medical institution at which the specimen is taken and the medical institution at which the personalized vaccine is administered as described above, the identification of the patient himself/herself may not be sufficiently checked. That is, the management may be insufficient, so that there is a risk that the vaccine may be mistakenly administered.

### SUMMARY

An object of the present disclosure is to provide an information processing system, an information processing method, and a program capable of solving at least one of the problems described above.

An aspect of the present disclosure for achieving the above-described object is an information processing system including:
biometric information acquisition means for acquiring biometric information of a patient;
authentication processing means for authenticating the patient based on the biometric information acquired by the biometric information acquisition means;
first storage means for storing the biometric information of the patient authenticated by the authentication processing means in association with identification information of the patient;
trace information generation means for generating signature information of the patient based on the biometric information of the patient authenticated by the authentication processing means and generating signed trace information containing the signature information;
trace information acquisition means for acquiring the signed trace information generated by the trace information generation means; and
verification processing means for performing a verification process on the signature information of the signed trace information acquired by the trace information acquisition means.

An aspect of the present disclosure for achieving the above-described object is an information processing method including:
acquiring biometric information of a patient;
authenticating the patient based on the acquired biometric information;
storing the biometric information of the authenticated patient in association with identification information of the patient;
generating signature information of the patient based on the biometric information of the authenticated patient and generating signed trace information containing the signature information;
acquiring the generated signed trace information; and
performing a verification process on the signature information of the acquired signed trace information.

An aspect of the present disclosure for achieving the above-described object is a program for causing a computer to perform:
a process for acquiring biometric information of a patient;
a process for authenticating the patient based on the acquired biometric information;
a process for storing the biometric information of the authenticated patient in association with identification information of the patient;
a process for generating signature information of the patient based on the biometric information of the authenticated patient and generating signed trace information containing the signature information;
a process for acquiring the generated signed trace information; and
a process for performing a verification process on the signature information of the acquired signed trace information.

An aspect of the present disclosure for achieving the above-described object is an information processing system including:
biometric information acquisition means for acquiring biometric information of a patient;
authentication processing means for authenticating the patient based on the biometric information acquired by the biometric information acquisition means;
first storage means for storing the biometric information of the patient authenticated by the authentication processing means in association with identification information of the patient;
trace information generation means for generating trace information of the patient containing the identification information of the patient authenticated by the authentication processing means;
trace information acquisition means for acquiring the trace information generated by the trace information generation means; and
verification processing means for performing a verification process for verifying matching between the identification information of the trace information acquired by the trace information acquisition means and identification information corresponding to the biometric information of the patient authenticated by the authentication processing means.

An aspect of the present disclosure for achieving the above-described object is an information processing method including:
acquiring biometric information of a patient;
authenticating the patient based on the acquired biometric information;
storing the biometric information of the authenticated patient in association with identification information of the patient;
generating trace information of the authenticated patient containing the identification information of the patient;
acquiring the generated trace information; and
performing a verification process for verifying matching between the identification information of the acquired trace information and identification information corresponding to the biometric information of the authenticated patient.

An aspect of the present disclosure for achieving the above-described object is a program for causing a computer to perform:
a process for acquiring biometric information of a patient;
a process for authenticating the patient based on the acquired biometric information;
a process for storing the biometric information of the authenticated patient in association with identification information of the patient;
a process for generating trace information of the authenticated patient containing the identification information of the patient;
a process for acquiring the generated trace information; and
a process for performing a verification process for verifying matching between the identification information of the acquired trace information and identification information corresponding to the biometric information of the authenticated patient.

### BRIEF DESCRIPTION OF DRAWINGS

The above and other aspects, features and advantages of the present disclosure will become more apparent from the following description of certain example embodiments when taken in conjunction with the accompanying drawings, in which:

FIG. 1 shows a schematic hardware configuration of an information processing system according to the present disclosure;

FIG. 2 is a block diagram showing a schematic system configuration of the information processing system according to the present disclosure;

FIG. 3 is a chart diagram of an information processing method according to the present disclosure;

FIG. 4A shows examples of images shown in a display of a terminal according to the present disclosure;

FIG. 4B shows examples of images shown in a display of a terminal according to the present disclosure;

FIG. 4C shows examples of images shown in a display of a terminal according to the present disclosure;

FIG. 5 is a block diagram showing a schematic system configuration of an information processing system according to the present disclosure;

FIG. 6 is a chart diagram of an information processing method according to the present disclosure; and

FIG. 7 is a chart diagram of an information processing method according to the present disclosure.

### EXAMPLE EMBODIMENT

### First Example Embodiment

For example, at a medical institution such as a hospital, a specimen such as blood or a part of a tumor of a cancer is taken from a patient, and based on the taken specimen, a so-called personalized vaccine or personalized therapeutic agent is prepared specifically for that patient through a plurality of institutions and companies. Then, the generated personalized vaccine or personalized therapeutic agent is administered to the same patient at a medical institution (which is the same as or different from the aforementioned medical institution).

As described above, in the process for generating and administering a personalized vaccine or a personalized therapeutic agent, a vaccine or a therapeutic agent generated based on a specimen taken from a patient needs to be administered to the same patient.

To enable this to be done, an information processing system according to this example embodiment is a system for ensuring that, for example, the process for generating and administering a personalized vaccine or a personalized therapeutic agent is correctly carried out by directly associating the patient himself/herself with the vaccine or therapeutic agent itself. Note that examples of personalized vaccines include a personalized cancer vaccine (PCV) and CAR-T.

That is, in the flow of administering a personalized vaccine or a personalized therapeutic agent, the information processing system according to this example embodiment checks the identification of a patient himself/herself more reliably through a biometric authentication and an electronic signature when a specimen is detected and when a vaccine or a therapeutic agent is administered. In this way, it is possible to, for example, greatly reduce the risk of mistakes being made in the administration of vaccines or therapeutic agents and mistakes in information of labels both of which would otherwise be caused due to insufficient checking (or confirmation) of the identification of the patient himself/herself.

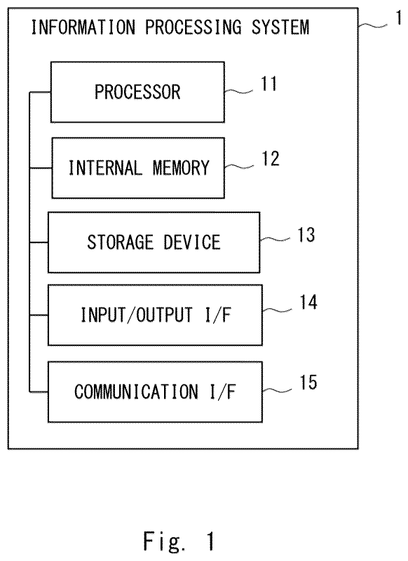

FIG. 1 shows a schematic hardware configuration of an information processing system according to the present disclosure. As shown in FIG. 1, the information processing system 1 according to this example embodiment has a hardware configuration of an ordinary computer including, for example, a processor 11 such as a CPU (Central Processing Unit) or a GPU (Graphics Processing Unit), an internal memory 12 such as a RAM (Random Access Memory) or a ROM (Read Only Memory), a storage device 13 such as an HDD (Hard Disk Drive) or an SSD (Solid State Drive), an input/output I/F 14 for connecting peripheral devices such as a display device, and a communication I/F 15 for communicating with external apparatuses.

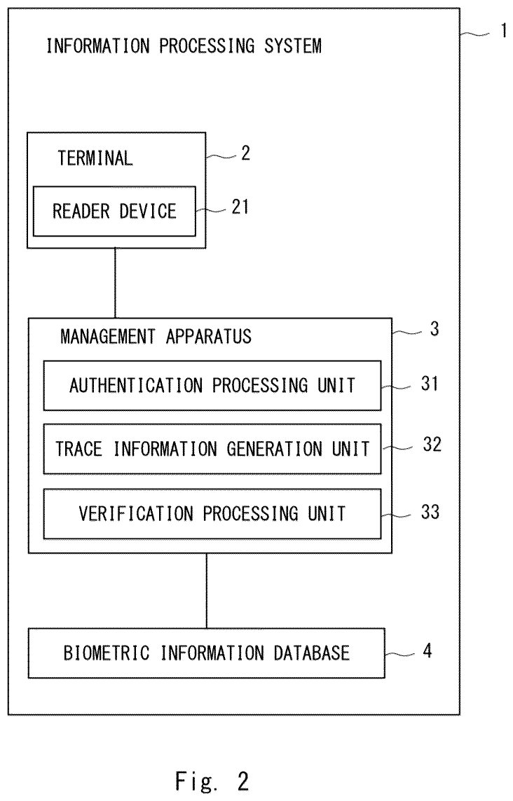

FIG. 2 is a block diagram showing a schematic system configuration of the information processing system according to the present disclosure. As shown in FIG. 2, the information processing system 1 according to this example embodiment includes a terminal 2, a management apparatus 3, and a biometric information database 4. Note that the terminal 2, the management apparatus 3, and the biometric information database 4 may be connected to each other through, for example, a wireless LAN (Local Area Network) or Wifi (Registered Trademark).

The terminal 2 is a specific example of the biometric information acquisition means. The terminal 2 is composed of a portable terminal that a user can operate, such as a smartphone, a tablet computer, and a PC (Personal Computer). The terminal 2 acquires biometric information of a patient. Examples of biometric information include the patient's face, iris(es), fingerprint(s), vein(s), voice print, palm print, and/or ear(s).

The terminal 2 acquires biometric information of a patient based on, for example, an image of the patient acquired by a camera or a voice of the patient acquired by a microphone. The terminal 2 transmits the acquired biometric information of the patient to the management apparatus 3.

The management apparatus 3 is an apparatus for managing the whole system and is configured as, for example, a server apparatus. The terminal 2 and the management apparatus 3 may be formed as one integrated apparatus. Either the terminal 2 or the management apparatus 3 may include the biometric information database 4 therein.

The management apparatus 3 includes an authentication processing unit 31 for performing a process for authenticating a patient, a trace information generation unit 32 for generating trace information for tracing, i.e., keeping track of, a specimen of the patient, and a verification processing unit 33 for performing a process for verifying signature information.

The authentication processing unit 31 is a specific example of the authentication processing means. The authentication processing unit 31 performs a process for authenticating a patient based on biometric information of the patient transmitted from the terminal 2. The authentication processing unit 31 performs the process for authenticating a patient by, for example, comparing biometric information of the patient transmitted from the terminal 2 with pre-registered biometric information.

The authentication processing unit 31 may perform a process for authenticating a patient by combining a plurality of pieces of biometric information of the patient with each other. For example, the authentication processing unit 31 may perform a process for authenticating a patient based on information on the face of the patient and information on his/her fingerprint(s).

The authentication processing unit 31 stores the biometric information of the successfully authenticated patient in the biometric information database 4 in association with the identification information (ID information) of the patient.

The biometric information database 4 is a specific example of the first storage means. The biometric information database 4 is composed of, for example, the storage device 13.

The trace information generation unit 32 is a specific example of the trace information generation means. The trace information generation unit 32 generates signature information (electronic signature) of the patient based on the biometric information of the patient authenticated by the authentication processing unit 31. Further, the trace information generation unit 32 generates signed trace information containing the generated signature information.

The trace information includes, for example, a QR code (Registered Trademarks), URL (Uniform Resource Locator) link information, or a barcode, and contains arbitrary information that is displayed in a form that can be read by a reader device 21. The management apparatus 3 may output the signed trace information as a label by a printer or the like.

For example, the label of the signed trace information, e.g., the label on which the signed trace information is printed, is attached to a vial, a container, or the like in which a vaccine or a therapeutic agent generated based on the specimen of the patient is contained. In this way, it is possible to directly associate the patient himself/herself with the vaccine or therapeutic agent itself.

Since the above-described signed trace information is verified by the information processing system 1 according to this example embodiment before the vaccine or therapeutic agent is administered to the patient, a doctor or a nurse can confirm that the vaccine or therapeutic agent was generated from the specimen of that patient.

Note that in the case where it is desired to also trace intermediate processes during the process through which the vaccine or therapeutic agent is generated from the specimen of the patient, the signed trace information may also be attached to the specimen or an intermediate product (including an electronic file) generated from the specimen.

The verification processing unit 33 is a specific example of the verification processing means. The verification processing unit 33 performs a process for verifying the signature information of the signed trace information read by the reader device 21 of the terminal 2 based on the biometric information of the patient authenticated by the authentication processing unit 31.

The reader device 21 is a specific example of the trace information acquisition means. The reader device 21 is composed of, for example, a barcode reader or a QR code reader. Note that the reader device 21 is disposed inside the terminal 2 in the above-described example, but the configuration of the reader device 21 is not limited to this example. That is, the reader device 21 may be formed separately from the terminal 2.

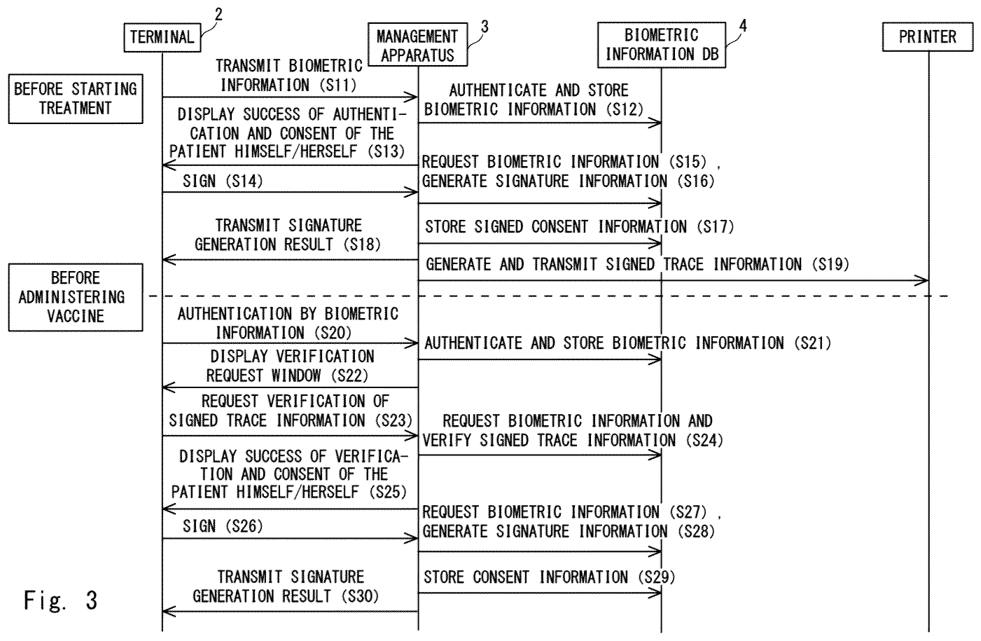

Next, an information processing method according to this example embodiment will be described with reference to FIG. 3. FIG. 3 is a chart diagram of an information processing method according to the present disclosure.

Firstly, before starting a treatment, the terminal 2 acquires biometric information of a patient and transmits the acquired biometric information of the patient to the management apparatus 3 (S11). The authentication processing unit 31 of the management apparatus 3 performs a process for authenticating the biometric information of the patient. When the authentication has succeeded, the authentication processing unit 31 of the management apparatus 3 stores the biometric information of the patient in the biometric information database (biometric information DB) 4 in association with identification information of the patient (S12).

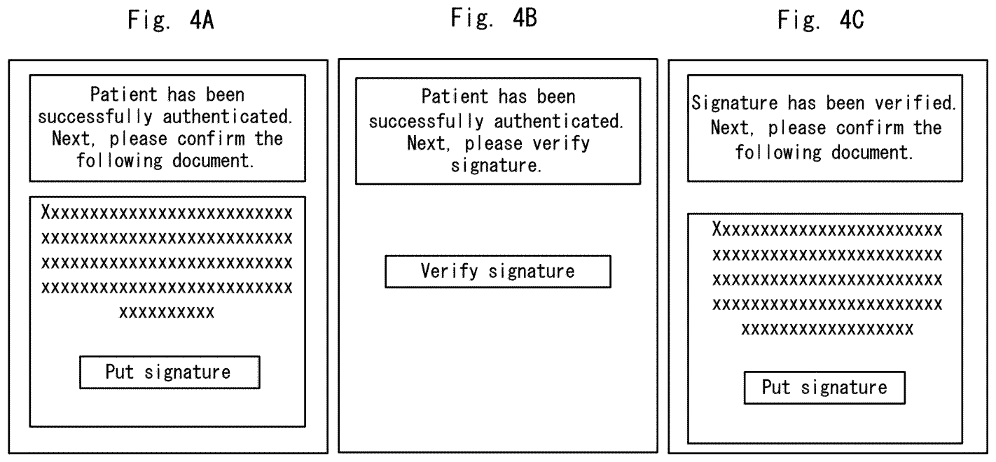

As shown in FIG. 4(a), for example, the management apparatus 3 makes, i.e., instructs, the terminal 2 display, on its screen, an authentication success window indicating that the authentication has succeeded and a patient consent window (consent information) for requesting the consent of the patient himself/herself as to, for example, taking a specimen from the patient (S13).

When the patient performs a signature operation such as instructing to “Put signature” on the screen of the terminal 2 (S14), the management apparatus 3 requests the biometric information database 4 to transmit biometric information corresponding to the identification information of that patient (S15). Note that the management apparatus 3 may omit (or skip) the above-described process for requesting the consent of the patient himself/herself (S13).

The biometric information database 4 transmits the biometric information to the management apparatus 3 in response to the above-described request. The trace information generation unit 32 of the management apparatus 3 generates signature information based on the biometric information (S16). Note that in the case where the authentication processing unit 31 performs a process for authenticating a patient by combining a plurality of pieces of biometric information with each other as described above, signature information may be generated for each of the pieces of biometric information based thereon.

The management apparatus 3 stores the signed consent information containing the generated signature information in the biometric information database 4 (S17). This signed consent information may be used later for auditing or the like.

Note that the consent information may contain, for example, in addition to the document of the consent of the patient himself/herself, a time stamp, a document in which the process for generating the vaccine is described, and identification information of the vaccine. The management apparatus 3 transmits a signature generation result indicating that the signature has been generated to the terminal 2 (S18).

The trace information generation unit 32 of the management apparatus 3 generates signed trace information containing signature information, and transmits the generated signed trace information to a printer (S19). The printer outputs the signed trace information as a label or the like. The label of the signed trace information, e.g., the label on which the signed trace information is printed, is attached to a vial, a container, or the like in which a vaccine or a therapeutic agent is contained.

Next, before the vaccine is administered to the patient, the terminal 2 acquires the biometric information of the patient and transmits the acquired biometric information of the patient to the management apparatus 3. The authentication processing unit 31 of the management apparatus 3 performs a process for authenticating the biometric information of the patient (S20). When the authentication has succeeded, the authentication processing unit 31 of the management apparatus 3 stores the biometric information of the patient in the biometric information database 4 in association with the identification information of the patient (S21).

The management apparatus 3 requests the terminal 2 to display a verification request window (S22). In response to this request, the terminal 2 displays, as the verification request window, for example, a window for requesting to verify the signature as shown in FIG. 4(b).

In the verification request window of the terminal 2, the patient performs an operation such as instructing to “Verify signature”. The reader device 21 of the terminal 2 reads the signed trace information attached to the vial or the like of the vaccine or therapeutic agent, and transmits the read signed trace information to the management apparatus 3. Then, the terminal 2 requests the management apparatus 3 to verify the signed trace information (S23).

In response to this request, the management apparatus 3 requests the biometric information corresponding to the identification information of the patient from the biometric information database 4. In response to this request, the biometric information database 4 transmits the biometric information corresponding to the identification information of the patient to the management apparatus 3. The verification processing unit 33 of the management apparatus 3 performs a process for verifying the signature information of the signed trace information based on the transmitted biometric information (S24).

When the verification of the signature information has succeeded, the verification processing unit 33 of the management apparatus 3 requests the terminal 2 to display, for example, a verification success window indicating that the verification of the signature information has succeeded and a pre-administration patient consent window (consent information) for requesting the consent of the patient himself/herself in regard to the administration of the vaccine as shown in FIG. 4(c) (S25). The terminal 2 displays the verification success window and the pre-administration patient consent window in response to this request.

When the patient performs a signature operation such as instructing to “Put signature” on the screen of the terminal 2 (S26), the management apparatus 3 requests the biometric information database 4 to transmit biometric information corresponding to the identification information of that patient (S27). Note that the management apparatus 3 may omit (or skip) the above-described process for requesting the consent of the patient himself/herself (S25).

In response to this request, the biometric information database 4 transmits the biometric information corresponding to the identification information of the patient to the management apparatus 3. The trace information generation unit 32 of the management apparatus 3 generates signature information based on the biometric information (S28).

The management apparatus 3 stores the signed consent information containing the signature information in the biometric information database 4 (S29). The management apparatus 3 transmits a signature generation result indicating that the signature has been generated to the terminal 2 (S30).

### Second Example Embodiment

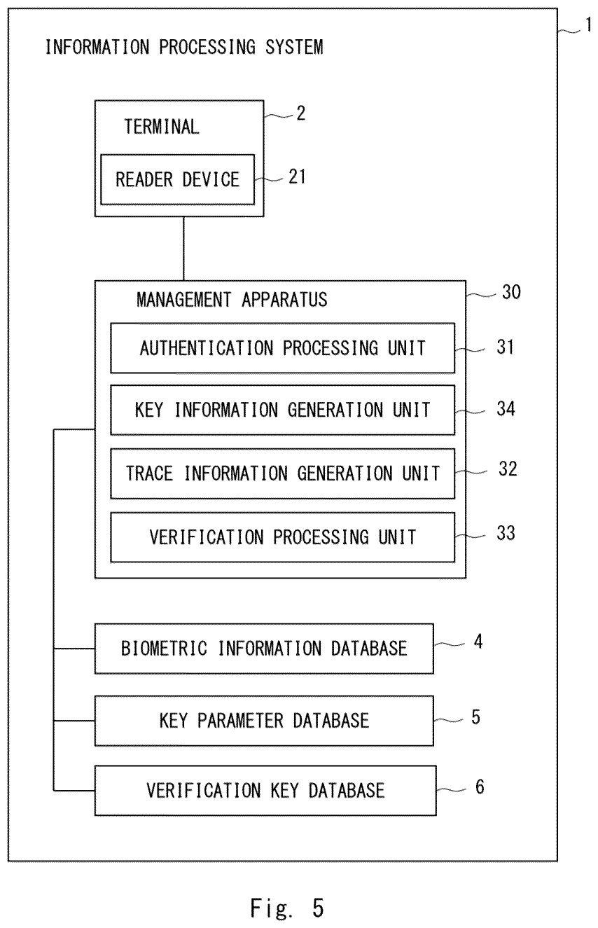

FIG. 5 is a block diagram schematically showing a system configuration of an information processing system according to the present disclosure. The information processing system 20 according to this example embodiment further includes, in addition to the configuration of the above-described first example embodiment, a verification key database 5 and a key parameter database 6.

The verification key database 5 and the key parameter database 6 are a specific example of the second storage means. The verification key database 5 and the key parameter database 6 are composed of, for example, a storage device(s).

Note that at least two of the biometric information database 4, the verification key database 5, and the key parameter database 6 may be combined with each other and thereby formed as one integrated apparatus. For example, the verification key database 5 and the key parameter database 6 may be formed as one integrated apparatus.

Further, the management apparatus 30 may include at least one of the biometric information database 4, the verification key database 5 and the key parameter database 6, and may conceivably have a configuration in which these components are arbitrarily combined with each other.

The management apparatus 30 according to this example embodiment further includes, in addition to the configuration of the above-described first example embodiment, a key information generation unit 34. The key information generation unit 34 is a specific example of the key information generation means. The key information generation unit 34 generates key parameter information for generating signature information of a patient and key verification information for verifying the signature information of the patient.

The key information generation unit 34 stores the generated key parameter information in the key parameter database 6 in association with the identification information of the patient. Further, the key information generation unit 34 also stores the generated verification key information in the verification key database 5 in association with the identification information of the patient.

The trace information generation unit 32 generates signature information based on the key parameter information and the biometric information. In this example embodiment, as described above, a signature key is generated in a state in which the signature key is secretly shared between the key parameter information (shared key) and the biometric information. In this way, the signature cannot be generated unless there are both the shared key and the biometric information, so that the protection against the leakage of the key can be improved.

The trace information generation unit 32 generates signed trace information containing the generated signature information. The verification processing unit 33 performs a process for verifying the signature information of the signed trace information based on the key verification information corresponding to the identification information of the patient authenticated by the authentication processing unit 31.

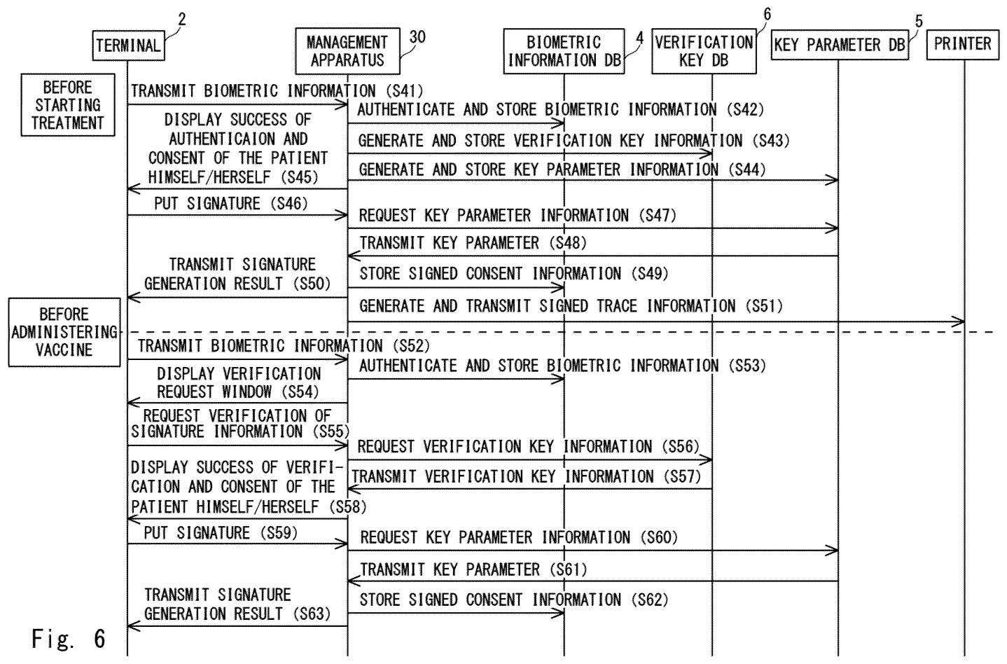

Next, an information processing method according to this example embodiment will be described with reference to FIG. 6. FIG. 6 is a chart diagram of an information processing method according to the present disclosure.

Firstly, before starting a treatment, the terminal 2 acquires biometric information of a patient and transmits the acquired biometric information of the patient to the management apparatus 30 (S41). The authentication processing unit 31 of the management apparatus 30 performs a process for authenticating the biometric information of the patient. When the authentication has succeeded, the authentication processing unit 31 of the management apparatus 30 stores the biometric information of the patient in the biometric information database (biometric information DB) 4 in association with identification information of that patient (S42).

The key information generation unit 34 of the management apparatus 30 generates verification key information, associates the generated verification key information with the identification information of the patient, and stores them in the verification key database (verification key DB) 5 (S43). Further, the key information generation unit 34 of the management apparatus 3 generates key parameter information, associates the generated key parameter information with the identification information of the patient, and stores them in the key parameter database (key parameter DB) 6 (S44).

As shown in FIG. 4(a), for example, the management apparatus 30 requests the terminal 2 to display, on its screen, an authentication success window indicating that the authentication has succeeded and a patient consent window (consent information) for requesting the consent of the patient himself/herself as to, for example, taking a specimen from the patient (S45). The terminal 2 displays an authentication success window and a patient consent window in response to this request.

When the patient performs a signature operation such as instructing to “Put signature” on the screen of the terminal 2 (S46), the management apparatus 3 requests the key parameter database 6 to transmit key parameter information corresponding to the identification information of that patient (S47). Note that the management apparatus 3 may omit (or skip) the above-described process for requesting the consent of the patient himself/herself (S45).

The key parameter database 6 transmits the key parameter information to the management apparatus 30 in response to this request (S48). The trace information generation unit 32 of the management apparatus 30 generates signature information based on the key parameter information and the biometric information corresponding to the identification information of the patient.

The trace information generation unit 32 of the management apparatus 30 stores the signed consent information containing the signature information in the biometric information database 4 (S49). The management apparatus 3 transmits a signature generation result indicating that the signature has been generated to the terminal 2 (S50).

The trace information generation unit 32 of the management apparatus 30 generates signed trace information containing signature information and transmits the generated signed trace information to a printer (S51). The printer outputs the signed trace information as a label or the like. The label of the signed trace information, e.g., the label on which the signed trace information is printed, is attached to a vial, a container, or the like in which a vaccine or a therapeutic agent is contained.

Next, before the vaccine is administered to the patient, the terminal 2 acquires the biometric information of the patient and transmits the acquired biometric information of the patient to the management apparatus 30 (S52). The authentication processing unit 31 of the management apparatus 30 performs a process for authenticating the biometric information of the patient.

When the authentication has succeeded, the authentication processing unit 31 of the management apparatus 30 stores the biometric information of the patient in the biometric information database 4 in association with the identification information of the patient (S53).

The management apparatus 30 requests the terminal 2 to display a verification request window (S54). In response to this request, the terminal 2 displays, as the verification request window, for example, information for requesting to verify the signature as shown in FIG. 4(b).

In the verification request window of the terminal 2, the patient performs an operation such as instructing to “Verify signature”. The reader device 21 of the terminal 2 reads the signature information of the signed trace information attached to the vial or the like of the vaccine or therapeutic agent, and requests the management apparatus 30 to verify the signature information (S55).

In response to this request, the management apparatus 30 requests verification key information corresponding to the identification information of the patient from the verification key database 5 (S56).

The verification key database 5 transmits the verification key information to the management apparatus 30 in response to this request (S57). The verification processing unit 33 of the management apparatus 30 performs a process for verifying the signature information of the signed trace information based on the verification key information.

When the verification of the signature information has succeeded, the management apparatus 30 requests the terminal 2 to display, for example, a verification success window indicating that the verification of the signature information has succeeded and a pre-administration patient consent window (consent information) for requesting the consent of the patient himself/herself in regard to the administration of the vaccine as shown in FIG. 4(c) (S58). The terminal 2 displays the verification success window and the pre-administration patient consent window in response to this request.

When the patient performs a signature operation such as instructing to “Put signature” on the screen of the terminal 2 (S59), the management apparatus 30 requests the key parameter database 6 to transmit the key parameter information corresponding to the identification information of that patient (S60). Note that the management apparatus 30 may omit (or skip) the above-described process for requesting the consent of the patient himself/herself (S58).

The key parameter database 6 transmits the key parameter information to the management apparatus 30 in response to this request (S61). The trace information generation unit 32 of the management apparatus 30 generates signature information based on the key parameter information and the biometric information corresponding to the identification information of the patient.

The management apparatus 30 stores the signed consent information containing the signature information in the biometric information database 4 (S62). The management apparatus 30 transmits a signature generation result indicating that the signature has been generated to the terminal 2 (S63).

### Third Example Embodiment

An information processing system 1 according to this example embodiment has a configuration similar to that of the above-described information processing system 1 according to the first example embodiment. However, the trace information generation unit 32 according to the above-described first example embodiment generates signature information of a patient based on the biometric information of the patient authenticated by the authentication processing unit 31, and generates signed trace information containing the signature information. In contrast, the trace information generation unit 32 according to this example embodiment differs from the above-described example embodiment because it generates, for a patient who has been authenticated based on his/her biometric information, trace information of the patient containing identification information of the patient.

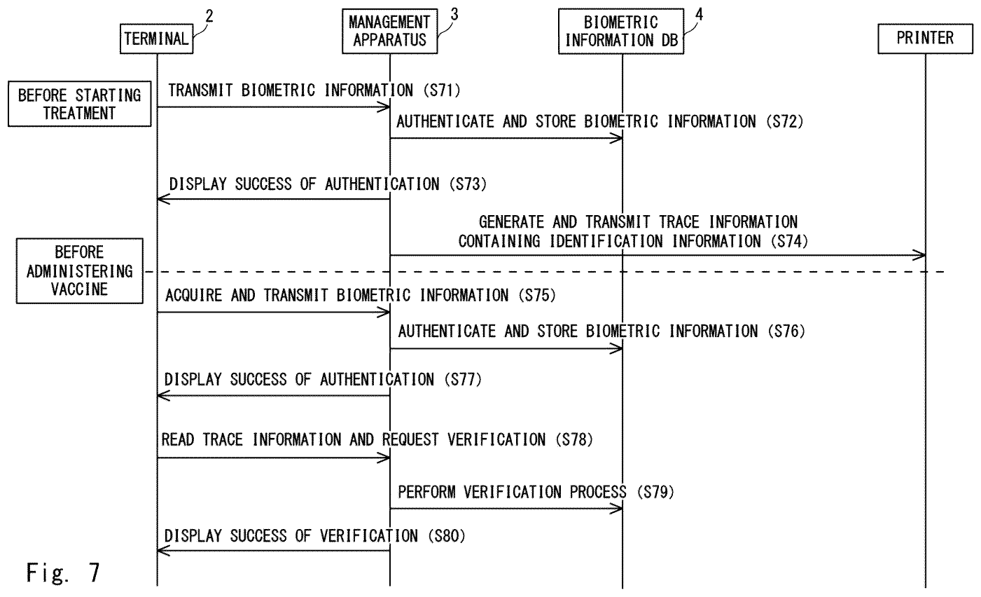

FIG. 7 is a chart diagram of an information processing method according to the present disclosure. Firstly, before starting a treatment, the terminal 2 acquires biometric information of a patient and transmits the acquired biometric information of the patient to the management apparatus 3 (S71).

The authentication processing unit 31 of the management apparatus 3 performs a process for authenticating the biometric information of the patient. When the authentication has succeeded, the authentication processing unit 31 of the management apparatus 3 stores the biometric information of the patient in the biometric information database 4 in association with identification information of the patient (S72).

The authentication processing unit 31 of the management apparatus 3 requests the terminal 2 to display, for example, an authentication success window indicating that the authentication has succeeded (S73). The terminal 2 displays the authentication success window in response to this request.

The trace information generation unit 32 of the management apparatus 3 generates trace information containing identification information of the patient and transmits the generated trace information to a printer (S74). The printer outputs the trace information as a label or the like. The label of the signed trace information, e.g., the label on which the trace information is printed, is attached to a vial, a container, or the like in which a vaccine or a therapeutic agent is contained.

Next, before the vaccine is administered to the patient, the terminal 2 acquires biometric information of the patient and transmits the acquired biometric information of the patient to the management apparatus 3 (S75). The authentication processing unit 31 of the management apparatus 3 performs a process for authenticating the biometric information of the patient. When the authentication has succeeded, the authentication processing unit 31 of the management apparatus 3 stores the biometric information of the patient in the biometric information database 4 in association with the identification information of the patient (S76).

The authentication processing unit 31 of the management apparatus 3 requests the terminal 2 to display, for example, an authentication success window indicating that the authentication has succeeded (S77). The terminal 2 displays the authentication success window in response to this request.

The reader device 21 of the terminal 2 reads the trace information attached to the vial or the like of the vaccine or therapeutic agent, and requests the management apparatus 3 to verify the trace information (S78).

The verification processing unit 33 of the management apparatus 3 performs a verification process for verifying matching between the identification information of the trace information read by the reader device 21 of the terminal 2 and identification information corresponding to the biometric information of the patient authenticated by the authentication processing unit 31 (S79).

When the verification has succeeded, the verification processing unit 33 of the management apparatus 3 requests the terminal 2 to display a verification success window indicating that the verification has succeeded (S80). The terminal 2 displays the verification success window in response to this request.

In the present disclosure, for example, the processes shown in FIG. 3, 6 or 7 may be implemented by having a processor execute a computer program.

The program may be stored and supplied to the computer using various types of non-transitory computer readable media. Non-transient computer readable media include various types of tangible storage media. Examples of non-transitory computer readable media include magnetic recording media (e.g., flexible disk, magnetic tape, hard disk drive), magneto-optical recording media (e.g., magneto-optical disks), CD-ROM (Read Only Memory), CD-R, CD-R/W, and semiconductor memory (e.g., mask ROM, programmable ROM (PROM), erasable PROM (EPROM), flash ROM, random access memory (RAM)).

The program may be provided to a computer using any type of transitory computer readable media. Examples of transitory computer readable media include electrical signals, optical signals, and electromagnetic waves. Transitory computer readable media can provide the program to a computer via a wired communication line (e.g., electric wires, and optical fibers) or a wireless communication line.

Each of the components constituting the information processing system 1 or 20 according to any of the above-described embodiments is, in addition to being able to be implemented by the program, able to be partially or entirely implemented by dedicated hardware such as an ASIC (Application Specific Integrated Circuit) or an FPGA (Field-Programmable Gate Array).

Although the present disclosure is described above with reference to example embodiments, the present disclosure is not limited to the above-described example embodiments. Various modifications that can be understood by those skilled in the art can be made to the configuration and details of the present disclosure within the scope and spirit of the disclosure. Further, the example embodiments may be combined with one another as appropriate.

Each of the drawings is merely an example to illustrate one or more embodiments. Each of the drawing is not associated with only one specific embodiment, but may be associated with one or more other embodiments. As will be understood by those skilled in the art, various features or steps described with reference to any one of the drawings may be combined with features or steps shown in one or more other drawings in order to create, for example, an embodiment that is not explicitly shown in the drawings or described in the specification. Not all of the features or steps shown in any one of the drawings to describe an embodiment are necessarily indispensable, and some features or steps may be omitted. The order of steps in any of the drawings may be changed as appropriate.

The whole or part of the example embodiments disclosed above can be described as, but not limited to, the following supplementary notes.

### Supplementary Note 1

An information processing system comprising: biometric information acquisition means for acquiring biometric information of a patient;
authentication processing means for authenticating the patient based on the biometric information acquired by the biometric information acquisition means;
first storage means for storing the biometric information of the patient authenticated by the authentication processing means in association with identification information of the patient;
trace information generation means for generating signature information of the patient based on the biometric information of the patient authenticated by the authentication processing means and generating signed trace information containing the signature information;
trace information acquisition means for acquiring the signed trace information generated by the trace information generation means; and
verification processing means for performing a verification process on the signature information of the signed trace information acquired by the trace information acquisition means.

### Supplementary Note 2

The information processing system described in Supplementary note 1, further comprising:
key information generation means for generating key verification information for verifying the signature information of the patient and key parameter information for generating the signature information; and
second storage means for storing the key verification information and the key parameter information generated by the key information generation means in association with the biometric information of the authenticated patient and the identification information of the patient, wherein
the trace information generation means generates the signature information based on the key parameter information and the biometric information corresponding to the identification information of the patient, and generates signed trace information containing the signature information, and
the verification processing means performs a process for verifying the signature information of the signed trace information acquired by the trace information acquisition means based on key verification information corresponding to the identification information of the patient authenticated by the authentication processing means.

### Supplementary Note 3

The information processing system described in Supplementary note 2, further comprising display means for displaying consent information for requesting consent of the patient, wherein
when the patient performs an operation for consenting for the consent information displayed on the display means, the trace information generation means generates the signature information based on the key parameter information generated by the key information generation means and the biometric information, and generates signed consent information containing the signature information.

### Supplementary Note 4

The information processing system described in Supplementary note 1, wherein the signed trace information generated by the trace information generation means is attached to a vaccine or a therapeutic agent generated based on a specimen taken from the patient.

### Supplementary Note 5

The information processing system described in Supplementary note 1, wherein the verification processing means performs the verification process before the vaccine or therapeutic agent is administered to the patient.

### Supplementary Note 6

The information processing system described in Supplementary note 5, wherein the vaccine or the therapeutic agent is a personalized vaccine or a personalized therapeutic agent for the patient.

### Supplementary Note 7

The information processing system described in Supplementary note 1, wherein the trace information is information displayed in a form that can be read by a reader device.

### Supplementary Note 8

An information processing method comprising:
acquiring biometric information of a patient;
authenticating the patient based on the acquired biometric information;
storing the biometric information of the authenticated patient in association with identification information of the patient;
generating signature information of the patient based on the biometric information of the authenticated patient and generating signed trace information containing the signature information;
acquiring the generated signed trace information; and
performing a verification process on the signature information of the acquired signed trace information.

### Supplementary Note 9

A program for causing a computer to perform:
a process for acquiring biometric information of a patient;
a process for authenticating the patient based on the acquired biometric information;
a process for storing the biometric information of the authenticated patient in association with identification information of the patient;
a process for generating signature information of the patient based on the biometric information of the authenticated patient and generating signed trace information containing the signature information;
a process for acquiring the generated signed trace information; and
process for performing a verification process on the signature information of the acquired signed trace information.

### Supplementary Note 10

An information processing system comprising:
biometric information acquisition means for acquiring biometric information of a patient;
authentication processing means for authenticating the patient based on the biometric information acquired by the biometric information acquisition means;
first storage means for storing the biometric information of the patient authenticated by the authentication processing means in association with identification information of the patient;
trace information generation means for generating trace information of the patient containing the identification information of the patient authenticated by the authentication processing means;
trace information acquisition means for acquiring the trace information generated by the trace information generation means; and
verification processing means for performing a verification process for verifying matching between the identification information of the trace information acquired by the trace information acquisition means and identification information corresponding to the biometric information of the patient authenticated by the authentication processing means.

### Supplementary Note 11

An information processing method comprising:
acquiring biometric information of a patient;
authenticating the patient based on the acquired biometric information;
storing the biometric information of the authenticated patient in association with identification information of the patient;
generating trace information of the authenticated patient containing the identification information of the patient;
acquiring the generated trace information; and
performing a verification process for verifying matching between the identification information of the acquired trace information and identification information corresponding to the biometric information of the authenticated patient.

### Supplementary Note 12

A program for causing a computer to perform:
a process for acquiring biometric information of a patient;
a process for authenticating the patient based on the acquired biometric information;
a process for storing the biometric information of the authenticated patient in association with identification information of the patient;
a process for generating trace information of the authenticated patient containing the identification information of the patient;
a process for acquiring the generated trace information; and
a process for performing a verification process for verifying matching between the identification information of the acquired trace information and identification information corresponding to the biometric information of the authenticated patient.

Some or all of the elements (e.g., structures and functions) described in Supplementary notes 2 to 7 that are dependent on Supplementary note 1 {e.g., an apparatus} can be dependent on Supplementary note 8 {e.g., a method} and Supplementary note 9 {e.g., a program} by the same dependency relationships as those in Supplementary notes 2 to 7.

Some or all of the elements described in any of the supplementary notes can be applied to various types of hardware, software, recording means for recording software, systems, and methods.

According to the present disclosure, it is possible to provide an information processing system, an information processing method, and a program capable of solving at least one of the problems described above.

The first and third example embodiments can be combined as desirable by one of ordinary skill in the art.

While the disclosure has been particularly shown and described with reference to example embodiments thereof, the disclosure is not limited to these example embodiments. It will be understood by those of ordinary skill in the art that various changes in form and details may be made therein without departing from the spirit and scope of the present disclosure as defined by the claims.

What is claimed is:

1. An information processing system comprising:
biometric information acquisition means for acquiring biometric information of a patient;
authentication processing means for authenticating the patient based on the biometric information acquired by the biometric information acquisition means;
first storage means for storing the biometric information of the patient authenticated by the authentication processing means in association with identification information of the patient;
trace information generation means for generating signature information of the patient based on the biometric information of the patient authenticated by the authentication processing means and generating signed trace information containing the signature information;
trace information acquisition means for acquiring the signed trace information generated by the trace information generation means; and
verification processing means for performing a verification process on the signature information of the signed trace information acquired by the trace information acquisition means.

2. The information processing system according to claim 1, further comprising:
key information generation means for generating key verification information for verifying the signature information of the patient and key parameter information for generating the signature information; and
second storage means for storing the key verification information and the key parameter information generated by the key information generation means in association with the biometric information of the authenticated patient and the identification information of the patient, wherein
the trace information generation means generates the signature information based on the key parameter information and the biometric information corresponding to the identification information of the patient, and generates signed trace information containing the signature information, and
the verification processing means performs a process for verifying the signature information of the signed trace information acquired by the trace information acquisition means based on key verification information corresponding to the identification information of the patient authenticated by the authentication processing means.

3. The information processing system according to claim 2, further comprising display means for displaying consent information for requesting consent of the patient, wherein
when the patient performs an operation for consenting for the consent information displayed on the display means, the trace information generation means generates the signature information based on the key parameter information generated by the key information generation means and the biometric information, and generates signed consent information containing the signature information.

4. The information processing system according to claim 1, wherein the signed trace information generated by the trace information generation means is attached to a vaccine or a therapeutic agent generated based on a specimen taken from the patient.

5. The information processing system according to claim 1, wherein the verification processing means performs the verification process before the vaccine or therapeutic agent is administered to the patient.

6. The information processing system according to claim 5, wherein the vaccine or the therapeutic agent is a personalized vaccine or a personalized therapeutic agent for the patient.

7. The information processing system according to claim 1, wherein the trace information is information displayed in a form that can be read by a reader device.

8. An information processing method comprising:
acquiring biometric information of a patient;
authenticating the patient based on the acquired biometric information;
storing the biometric information of the authenticated patient in association with identification information of the patient;
generating signature information of the patient based on the biometric information of the authenticated patient and generating signed trace information containing the signature information;
acquiring the generated signed trace information; and
performing a verification process on the signature information of the acquired signed trace information.

9. An information processing system comprising:
biometric information acquisition means for acquiring biometric information of a patient;
authentication processing means for authenticating the patient based on the biometric information acquired by the biometric information acquisition means;
first storage means for storing the biometric information of the patient authenticated by the authentication processing means in association with identification information of the patient;
trace information generation means for generating trace information of the patient containing the identification information of the patient authenticated by the authentication processing means;
trace information acquisition means for acquiring the trace information generated by the trace information generation means; and
verification processing means for performing a verification process for verifying matching between the identification information of the trace information acquired by the trace information acquisition means and identification information corresponding to the biometric information of the patient authenticated by the authentication processing means.
